(19) United States
(12) Patent Application Publication
(10) Pub. No.: US 2023/0260117 A1
(43) Pub. Date: Aug. 17, 2023

(54) INFORMATION PROCESSING SYSTEM, ENDOSCOPE SYSTEM, AND INFORMATION PROCESSING METHOD

(71) Applicant: OLYMPUS CORPORATION, Tokyo (JP)

(72) Inventors: Hiroshi MATSUZAKI, Tokyo (JP); Ryosuke MURATA, Tokyo (JP)

(73) Assignee: OLYMPUS CORPORATION, Tokyo (JP)

(21) Appl. No.: 18/140,106

(22) Filed: Apr. 27, 2023

Related U.S. Application Data
(63) Continuation of application No. PCT/JP2020/041659, filed on Nov. 9, 2020.

(51) Int. Cl. G06T 7/00; G16H 50/20; G06V 10/764; G06V 10/141; G06T 3/40

(52) U.S. Cl. CPC G06T 7/0012; G16H 50/20; G06V 10/764; G06V 10/141; G06T 3/40; G06T 2207/20081; G06T 2207/30096; G06V 2201/07; G06T 2207/10068

(57) ABSTRACT

The information processing system includes: a memory storing a first trained model and a second trained model; and a processor. The first trained model is trained to perform a detection process on a first training image at a normal magnification. The second trained model is trained to perform a diagnosis process on a second training image at a greater magnification. To the processor, magnification change information acquired by an operation device of an endoscope system that changes magnification of a target image is input. When the magnification change information indicates the normal magnification, the processor performs the detection process of detecting a lesion from the target image through processing based on the first trained model, and when the magnification change information indicates the greater magnification, the processor performs the diagnosis process of diagnosing a type of the lesion from the target image through processing based on the second trained model.

### CROSS REFERENCE TO RELATED APPLICATIONS

This application is a continuation of International Patent Application No. PCT/JP2020/041659, having an international filing date of Nov. 9, 2020, which designated the United States, the entirety of which is incorporated herein by reference.

### BACKGROUND OF THE INVENTION

There has been known a technique of detecting a class, a position, or an area of an object captured in an image using machine learning. This technique enables detailed recognition of an object area captured in the image and is useful, for example, for automatic segmentation of the object, image processing such as image quality improvement to the object area, or focusing on an object position. In the medical field, the above technique can be used to recognize a lesion area captured in the image to thereby reduce the burden on a physician in interpretation of radiogram. Also from such point of view, the above technique is useful. A conventional technique for such diagnostic support is disclosed, for example, in Japanese Unexamined Patent Publication No. 2016-87370. In Japanese Unexamined Patent Publication No. 2016-87370, an abnormal portion in an image of a large-intestine mucosa is detected, determination is performed as to which of five types of pit patterns of a large-intestine polyp the abnormal portion correspond to, using a trained model that has been subjected to machine learning, and a result of the determination is presented to the physician.

### SUMMARY OF THE INVENTION

In accordance with one of some aspect, there is provided a n information processing system comprising: a memory storing a first trained model and a second trained model; and a processor configured to perform a detection process and a diagnosis process, the detection process being a process of detecting a lesion from a target image through processing based on the first trained model, the target image being captured by an imaging device of an endoscope system and input to the processor, the diagnosis process being a process of diagnosing a type of the lesion from the target image through processing based on the second trained model, wherein the first trained model is a trained model that has been trained, using a first training image having a normal magnification, to perform the detection process on the first training image, the second trained model is a trained model that has been trained, using a second training image that has a greater magnification than the normal magnification, to perform the diagnosis process on the second training image, and magnification change information being input to the processor from an operation device of the endoscope system configured to change magnification of the target image, the processor is configured to perform the detection process on the target image when the magnification change information indicates the normal magnification, and perform the diagnosis process on the target image when the magnification change information indicates the greater magnification.

In accordance with one of some aspect, there is provided an endoscope system comprising: the information processing system as defined above; the imaging device; and the operation device.

In accordance with one of some aspect, there is provided an information processing method comprising: inputting a target image captured by an imaging device of an endoscope system and magnification change information acquired by an operation device of the endoscope system that changes magnification of the target image, performing a detection process when the magnification change information indicates a normal magnification, the detection process being a process of detecting a lesion from the target image through processing based on a first trained model that has been trained, using a first training image having the normal magnification, to detect the lesion from the first training image, and performing a diagnosis process when the magnification change information indicates a greater magnification than the normal magnification, the diagnosis process being a process of diagnosing a type of the lesion from the target image through processing based on a second trained model that has been trained, using a second training image having the greater magnification, to diagnose a type of the lesion from the second training image.

### BRIEF DESCRIPTION OF THE DRAWINGS

FIG. 1 is a first configuration example of an endoscope system.

FIG. 2 is a second configuration example of an endoscope system.

FIG. 3 is a first example of processing performed by a processing device.

FIG. 4 is an explanatory diagram of the processing performed by the processing device of the first example.

FIG. 5 is a configuration example of a learning device.

FIG. 6 is a first detailed example of a normal-magnification detection process.

FIG. 7 is a second detailed example of a normal-magnification detection process.

FIG. 8 is an explanatory diagram of the processing performed by the normal-magnification detection process of the second detailed example.

FIG. 9 is a detailed example of a greater-magnification diagnosis process.

FIG. 10 is a flowchart of the processing performed by the processing device.

FIG. 11 is a flowchart of the processing performed by the processing device.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Endoscope System and Information Processing System

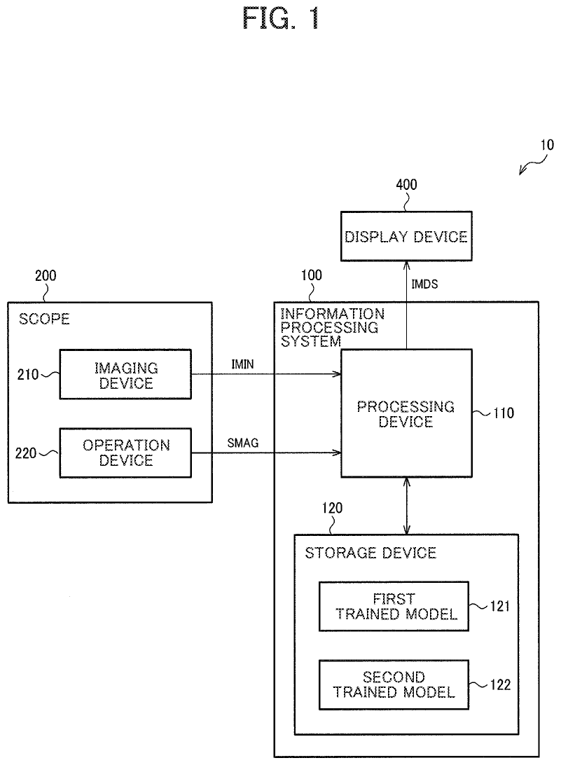

FIG. 1 shows a first configuration example of an endoscope system 10. The endoscope system 10 includes an information processing system 100, a scope 200, and a display device 400.

The scope 200 includes an imaging device 210 and an operation device 220. The scope 200 is inserted into a living body, captures an image inside the living body with the imaging device 210 provided at its tip, and transmits data of the image to the information processing system 100. The image captured by the imaging device 210 will be referred to as a target image IMIN. The target image IMIN refers to, for example, each frame image of a video captured by the scope 200.

The imaging device 210 is a device that captures images inside the living body. For example, the imaging device 210 includes an optical system that forms an image of a subject and an image sensor that photoelectrically converts the formed image. The optical system may be configured to have a fixed or variable optical imaging magnification.

The operation device 220 is a device for allowing a user of the endoscope system 10 to perform an operation input to the endoscope system 10. The operation input corresponds to, for example, changing the magnification, switching an observation mode, or the like. The operation device 220 is constituted, for example, by buttons, dials, or the like. Note that the operation device 220 may be a touch panel or the like provided in the display device 400, or a pointing device, a keyboard, or the like connected to a control device of the endoscope system 10.

When the optical system of the imaging device 210 is configured to have a variable imaging magnification, the imaging magnification of the optical system may be changed by an operation input of the operation device. Alternatively, digital zooming or the like may be applied to perform display at a displaying magnification corresponding to the operation input. As shown in FIG. 1, a signal or data indicating the magnification input from the operation device 220 is referred to as magnification change information SMAG. The observation mode is a mode according to the type of illumination light or whether or not to involve staining.

As the illumination light, white light and special light can be assumed, for example. The white light, which is also called normal light, is continuous spectrum light in a visible light wavelength region generated by, for example, a xenon lamp or the like, or light generated by an RGB3 light source such as an LED, a laser, or the like. The special light is so-called narrow band light and has a spectrum constituted by one or more narrow bands. The width of each narrow band is narrower than a region width of the visible light wavelength region. For example, the width of the narrow band belonging to a blue wavelength region is narrower than the region width of the wavelength range in that blue wavelength region. As special light observation, narrow band imaging (NBI) using blue narrow band light and green narrow band light can be assumed, for example.

Staining is a technique to stain a mucosal surface or a mucosal cell by spraying an agent on the mucosal surface to highlight irregularities on the mucosa, structure of the mucosal cell, or the like. As the agent, indigo blue, crystal violet, or the like are assumed, for example.

The information processing system 100 includes a processing device 110 and a storage device 120. The processing device 110 performs an image recognition process using machine learning on the target image IMIN, generates a display image IMDS from the result of the image recognition process and the target image IMIN, and outputs the generated display image IMDS to the display device 400. The display device 400 is a display unit, such as a liquid crystal display device or the like, for example, and displays the display image IMDS. The storage device 120 stores a first trained model 121 and a second trained model 122 that have been subjected to machine learning in advance using training data. The processing device 110 performs switching between a detection process based on the first trained model 121 and a diagnosis process based on the second trained model 122 in accordance with the magnification change information SMAG. Details of the machine learning, the detection process, and the diagnosis process are described below.

The hardware constituting the processing device 110 is a general-purpose processor such as a central processing unit (CPU), a graphical processing unit (GPU), a digital signal processor (DSP), or the like. In this case, the storage device 120 stores a program describing an inference algorithm and parameters used in the inference algorithm as the first trained model 121 and the second trained model 122. Alternatively, the processing device 110 may be a dedicated processor including the inference algorithm as a hardware. The dedicated processor is, for example, an application specific integrated circuit (ASIC), a field programmable gate array (FPGA), or the like. In this case, the storage device 120 stores parameters used in the inference algorithm as the first trained model 121 and the second trained model 122. The storage device 120 is a storage unit such as a semiconductor memory, a hard disk drive, an optical disk drive, or the like, for example. The semiconductor memory is a RAM, a ROM, a non-volatile memory, or the like.

As the inference algorithm, a neural network can be employed, for example. The parameters correspond to weight coefficients of inter-node connections in the neural network. The neural network includes an input layer to which the image data is input, an intermediate layer that performs arithmetic processing on the data that has been input through the input layer, and an output layer that outputs a recognition result on the basis of an arithmetic operation result output from the intermediate layer. As a neural network used for image recognition processing, a convolutional neural network (CNN) is preferable. However, not only the CNN, various neural network technologies can be employed. Further, the inference algorithm is not limited to the neural network, and various machine learning technologies used for image recognition can be employed.

In the first configuration example, the information processing system 100 concurrently serves as a control device for the endoscope system 10. That is, the processing device 110 controls respective devices of the endoscope system 10. For example, the processing device 110 performs imaging timing control, image processing to captured images, control of respective devices in response to operation inputs, and the like.

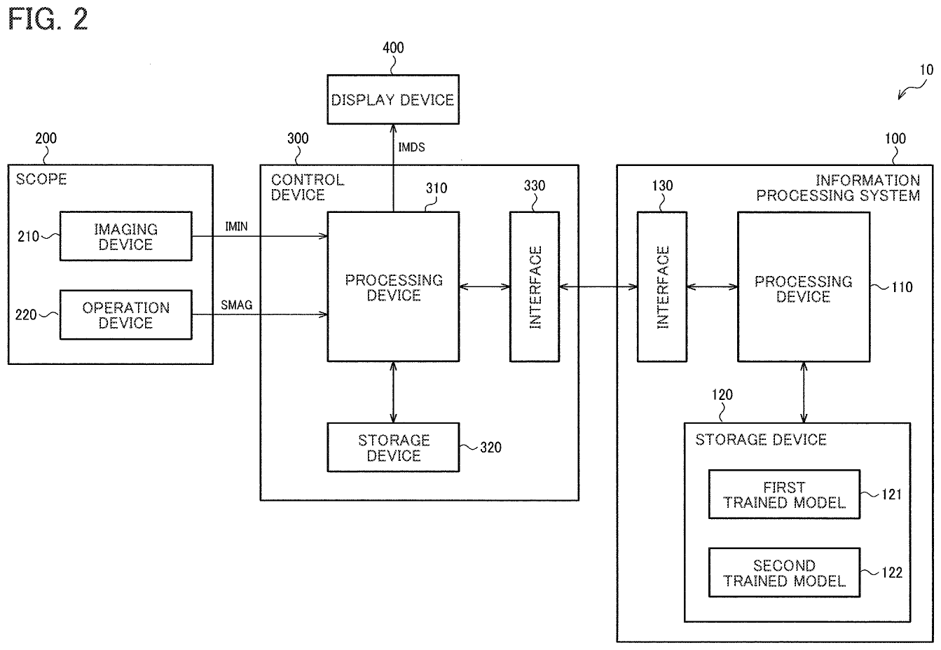

FIG. 2 shows a second configuration example of the endoscope system 10. In the second configuration example, the endoscope system 10 further includes a control device 300, and is configured to include an information processing system 100 separately from the control device 300.

The control device 300 includes a processing device 310, a storage device 320, and an interface 330. The processing device 310 is a processor such as a CPU and controls respective devices of the endoscope system 10. The storage device 320, which is a storage unit such as a semiconductor memory or the like, performs storing of operational setting information for the endoscope system 10, recording of image data, operations as a working memory for the processing device 310, or the like. The interface 330 is a communication interface that communicably connects the control device 300 to the information processing system 100. The communication method of the interface 330 is wired communication such as a wired LAN or USB, wireless communication such as a wireless LAN, network communication via the Internet, or the like.

The information processing system 100 includes the processing device 110, the storage device 120, and the interface 130. The information processing system 100 may be an information processing device such as a PC, or a cloud system constituted by a plurality of information processing devices connected to a network.

The interface 130 is an interface communicatively connected to the interface 330. The processing device 310 of the control device 300 transmits the target image IMIN and the magnification change information SMAG to the processing device 110 of the information processing system 100 via the interface 330 and the interface 130. The processing device 110 performs the detection process or the diagnosis process using the received target image IMIN and the magnification change information SMAG, and transmits the result of the process to the processing device 310 of the control device 300 via the interface 130 and the interface 330. The processing device 310 generates the display image IMDS from the received result and the target image IMIN.

### 2. Processing Device

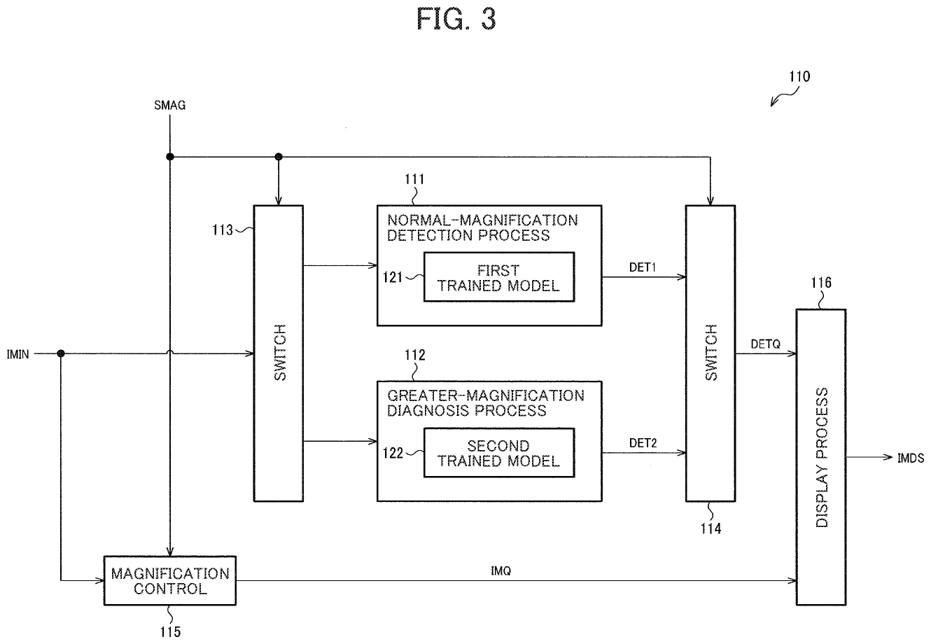

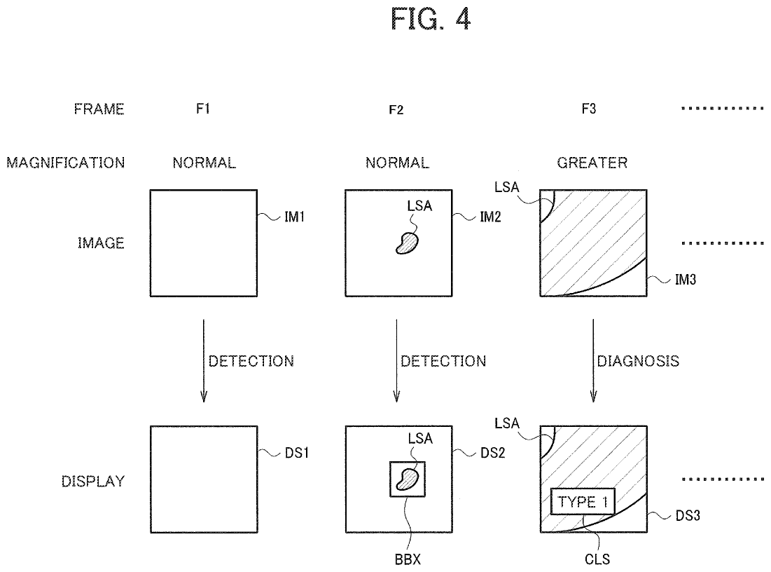

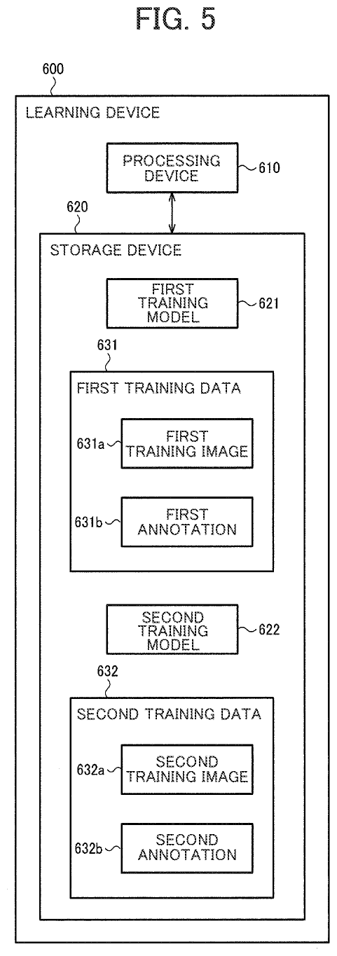

FIG. 3 shows a first example of the processing performed by the processing device 110. Further, FIG. 4 shows an explanatory diagram of the processing performed by the processing device 110 of the first example. Further, FIG. 5 shows a configuration example of a learning device 600.

As shown in FIG. 3, a switch 113 performs processing of selecting either a normal-magnification detection process 111 or a greater-magnification diagnosis process 112 according to the magnification change information SMAG. The switch 113 outputs the target image IMIN to the normal-magnification detection process 111 when the magnification change information SMAG indicates the normal magnification, and outputs the target image IMIN to the greater-magnification diagnosis process 112 when the magnification change information SMAG indicates the greater magnification.

The target image IMIN is input from the imaging device 210 to the switch 113, and the magnification change information SMAG is input from the operation device 220 to the switch 113. Alternatively, the target image IMIN and the magnification change information SMAG may be stored temporarily in the storage device 120, and after completion of imaging of the inside of the living body, may be input from the storage device 120 to the switch 113. For example, in the storage device 120, a video is recorded and metadata for each frame image of the video is recorded, and the metadata contains the magnification change information SMAG.

The normal magnification is magnification used for roughly observing a wide area inside the living body, and is magnification used for so-called screening. The greater magnification is a magnification higher than the normal magnification, magnification used for observing the living body in detail in close proximity to the living body, and magnification used for so-called enlarged observation. The magnification means magnification of displaying a subject on the display screen. For example, when the greater magnification corresponds to 100 times the normal magnification, a subject of 1 mm on the display screen at the normal magnification is magnified to 100 mm on the display screen at the greater magnification. As mentioned above, the magnification may be changed optically or may be changed by image processing such as digital zooming or the like.

The normal-magnification detection process 111 is processing using the first trained model 121 and is processing of detecting a lesion from the target image IMIN. When the first trained model 121 is a neural network, the target image IMIN is input to an input layer of the neural network, and the neural network detects a position of the lesion from the target image IMIN and generates a detection position of the lesion and a score indicating certainty of the detection. An output layer of the neural network outputs, when the score exceeds a preset threshold value, the detection position as a detection result DET1.

The normal-magnification detection process 111 corresponds to a detector that performs an image recognition process called detection. As shown in FIG. 4, it is assumed that images are captured at the normal magnification in frames F1 and F2 and an image is captured at the greater magnification in a frame F3. To images IM1 and IM2 in the frames F1 and F2, the normal-magnification detection process 111 is applied. Since the image IM1 contains no lesion, the detection result is not displayed in a display image DS1, and the image IM1 is displayed as it is as the display image DS1. Since the image IM2 contains a lesion LSA, the normal-magnification detection process 111 generates a boundary box BBX indicating a position of the lesion LSA, and the boundary box BBX is superimposed on the image IM2 and displayed as the display image DS2. The boundary box BBX has a rectangular shape circumscribed by the lesion LSA.

The first trained model 121 has been trained in advance by the learning device 600 in FIG. 5. The learning device 600 is implemented by an information processing device such as a PC or the like, and includes a processing device 610 and a storage device 620. The processing device 610 is a processor such as a CPU or the like, and the storage device 120 is a storage unit such as a semiconductor memory, a hard disk drive, or the like. The storage device 620 stores a first training model 621 and first training data 631 and generates the first trained model 121 by being subjected to training of the first training model 621 by the processing device 610 using the first training data 631. The generated first trained model 121 is transferred to the storage device 120 of the information processing system 100.

The first training data 631 includes a number of first training images 631a and first annotations 631b attached to the respective first training images 631a. The first training image 631a is an image of the inside of the living body captured by the endoscope system 10 at the normal magnification. The physician or the like attaches the first annotation 631b to the first training image 631a. The first annotation 631b is a boundary box indicating the position of the lesion contained in the first training image 631a.

The greater-magnification diagnosis process 112 in FIG. 3 is processing that uses the second trained model 122 and processing of diagnosing the type of the lesion from the target image IMIN. When the second trained model 122 is a neural network, the target image IMIN is input to an input layer of the neural network, and the neural network classifies types of the lesion from the target image IMIN and generates a score indicating probability of being the type for the respective types. An output layer of the neural network outputs the type having the highest score as a diagnosis result DET2.

The greater-magnification diagnosis process 112 is a classifier that classifies images into classes or categories. As shown in FIG. 4, the greater-magnification diagnosis process 112 is applied to the image IM3 in the frame F3 captured at the greater magnification. The image IM3 is an image in which the lesion LSA of the image IM2 is captured at the greater magnification. The greater-magnification diagnosis process 112 classifies the type of the lesion LSA from the image IM3. For example, when the lesion LSA is determined to be a “type 1,” letters CLS of the “type 1” is superimposed on the image IM3 and displayed as the display image DS2.

The second trained model 122 has been trained in advance by the learning device 600 in FIG. 5. The storage device 620 stores a second training model 622 and a second training data 632 and generates the second trained model 122 by being subjected to training of the second training model 622 by the processing device 610 using the second training data 632. The generated second trained model 122 is transferred to the storage device 120 of the information processing system 100.

The second training data 632 includes a number of second training images 632a and second annotations 632b attached to the respective second training images 632a. The second training image 632a is an image in which the inside of the living body is captured by the endoscope system 10 at the greater magnification. The physician or the like attaches the second annotation 632b to the second training image 632a. The second annotations 632b are types of the lesions contained in the second training images 632a.

A switch 114 in FIG. 3 performs processing of selecting either the detection result DET1 or the diagnosis result DET2 according to the magnification change information SMAG. The switch 114 outputs the detection result DET1 as an image recognition result DETQ to a display process 116 when the magnification change information SMAG indicates the normal magnification, and outputs the diagnosis result DET2 as the image recognition result DETQ to the display process 116 when the magnification change information SMAG indicates the greater magnification.

Magnification control 115 is processing of controlling the magnification of the image according to the magnification change information SMAG. The magnification control 115 performs an enlargement process of the target image IMIN at the magnification indicated by the magnification change information SMAG, and outputs an image IMQ after the enlargement to the display process 116. The magnification control 115 outputs the target image IMIN as the image IMQ to the display process 116 when the magnification is one-power. Although an example of changing the displaying magnification by image processing has been described here, the imaging magnification of the imaging device 210 may be changed optically.

The display process 116 generates the display image IMDS from the image IMQ and the image recognition result DETQ, and outputs the generated display image IMDS to the display device 400. For example, the display process 116 generates the display image IMDS by overlaying the image IMQ with the image recognition result DETQ. Alternatively, the display process 116 may display the image recognition result DETQ together with the reliability of the image recognition result DETQ on the display image IMDS. In this case, the normal-magnification detection process 111 outputs the detection result together with a score of the detection result, and the greater-magnification diagnosis process 112 outputs the diagnosis result together with a score of the diagnosis result. The display process 116 displays these scores as reliabilities on the display image IMDS.

When the endoscope system 10 is for a digestive tract, for example, various lesions such as polyps, cancer, or inflammatory diseases caused in the mucosa of the digestive tract are assumed as lesions. Types of lesions are polyps, cancer, inflammatory diseases, or the like, which are further classified individually into lower classes or categories. Specifically, the types are typological types, malignancy grades, or the like that can be classified by features on the image such as microstructure, color, or the like.

Using pit pattern diagnosis of polyps with a large-intestine endoscope as an example, description will be made on flows of the diagnosis and the types of lesions. First, the physician sets illumination light to narrow band imaging (NBI) light and the magnification of the image to the normal magnification, and searches for a region in the large-intestine mucosa where a polyp is suspected to exist. At this time, the processing device 110 performs the normal-magnification detection process 111 and displays a position possibly being a polyp by the boundary box BBX.

The physician sets the magnification of the image to the greater magnification, enlarges and displays the region where a polyp is suspected to exist, and diagnoses the type of the polyp from a travelling pattern of microvessels in the mucosa, the structure of a ductal opening, the shape of the mucosal cell, or the like. The illumination light remains to be the narrow band imaging (NBI) light. At this time, the processing device 110 performs the greater-magnification diagnosis process 112 and displays the type of the polyp. The type of the polyp is a typological type classified according to tissue structure such as the travelling pattern of microvessels in the mucosa, the structure of the ductal opening, or the shape of the mucosal cell, or the depth to which the abnormal tissue reaches. The second trained model 122 is trained to enable recognition of the typological type of this polyp from the image.

When the physician observes the image, the observation may be performed for various purposes such as to roughly detect the position of the lesion, to diagnose the malignancy grade of the lesion, or the like. As appropriate for these purposes, the physician performs observation while incorporating observation at the normal magnification, observation at the greater magnification, or the like. Depending on the magnification of the image, organs, tissues, mucosal textures, or the like appear differently, and required diagnostic support differs depending on the purpose. Therefore, it is difficult to deal with all tasks with one kind of trained model.

In the present embodiment as described above, the information processing system 100 includes the storage device 120 and the processing device 110. The storage device 120 stores the first trained model 121 and the second trained model 122. The processing device 110 receives an input of the target image IMIN captured by the imaging device 210 of the endoscope system 10 and performs the detection process of detecting the lesion from the target image IMIN through processing based on the first trained model 121 and the diagnosis process of diagnosing the type of the lesion from the target image IMIN through processing based on the second trained model 122. The first trained model 121 is a trained model that has been trained, using the first training image 631a that has the normal magnification, to perform the detection process on the first training image 631a. The second trained model 122 is a trained model that has been trained, using the second training image 632a that has a greater magnification than the normal magnification, to perform the diagnosis process on the second training image 632a. To the processing device 110, the magnification change information SMAG acquired by the operation device 220 of the endoscope system 10 that changes the magnification of the target image IMIN is input. The processing device 110 performs the detection process on the target image IMIN when the magnification change information SMAG indicates the normal magnification, and performs the diagnosis process on the target image IMIN when the magnification change information SMAG indicates the greater magnification.

According to the present embodiment, in observation using an endoscope, the trained model used for the diagnostic support is switched between the observation at the normal magnification and the observation at the greater magnification. In other words, it is possible to prepare a trained model corresponding to appearance at the normal magnification of organs, tissues, mucosal textures, or the like and a trained model corresponding to appearance at the greater magnification of organs, tissues, mucosal textures, or the like, and to use these models in a switching manner. This makes it possible to perform diagnostic support capable of dealing with various tasks that combine the observation at the normal magnification with the observation at the greater magnification.

In the present embodiment, a normal image, which is the target image IMIN at the normal magnification, is an image captured under a first condition including at least one of white light condition, special light condition, and stained condition. The first trained model 121 is trained using the first training image 631a captured under the first condition. A special image, which is the target image IMIN at the greater magnification, is an image captured under a second condition including at least one of special light condition and stained condition, and an enlarged part of a field of view of the normal image. The second trained model 122 is trained using the second training image 632a captured under the second condition.

According to the present embodiment, the target image IMIN to be inferred at the normal magnification, and the first training image 631a used for training of the first trained model 121 are images captured under the same first condition. As a result, the first trained model 121 corresponding to the appearance at the normal magnification of organs, tissues, mucosal textures, or the like is generated, and the detection process using the generated first trained model 121 enables appropriate diagnostic support at the normal magnification. Similarly, the target image IMIN to be inferred at the greater magnification, and the second training image 632a used for training of the second trained model 122 are images captured under the same second condition. As a result, the second trained model 122 corresponding to the appearance at the greater magnification of organs, tissues, mucosal textures, or the like is generated, and the diagnosis process using the generated second trained model 122 enables appropriate diagnostic support at the normal magnification.

In the present embodiment, the target image IMIN at the normal magnification and the target image IMIN at the greater magnification are images captured under the special light condition and unstained condition. The greater magnification is between 50 times or more to less than 100 times the normal magnification at observation magnification on the display screen.

The special light is used in pit pattern diagnosis using the large-intestine endoscope. In the pit pattern diagnosis, an image with a texture state preferred for diagnosis is captured by setting the greater magnification to 50 times or more to less than 100 times the normal magnification. According to the present embodiment, in the pit pattern diagnosis using the large-intestine endoscope, it is possible to appropriately provide diagnostic support in the normal observation and diagnostic support in the enlarged observation.

Further, in the present embodiment, the spectrum of the special light has a peak belonging to a bandwidth of 390 nm to 445 nm and a peak belonging to a bandwidth of 530 nm to 550 nm.

The NBI is used in the pit pattern diagnosis using the large-intestine endoscope. The spectrum of the above-mentioned special light is the spectrum of the illumination light used in the NBI. According to the present embodiment, it is possible, in the NBI, to appropriately provide the diagnostic support in the normal observation and the diagnostic support in the enlarged observation. Using the NBI can clearly visualize textural features of the lesion and therefore can provide accurate diagnostic support.

Note that the special light is not limited to the illumination light used in the above-mentioned NBI. For example, the special light may be the illumination light used for linked color imaging (LCI), which combines a laser for white light and a laser for narrow-band light observation, or blue light imaging (BLI), which adjusts the emission ratio of two kinds of laser light.

Further, in the present embodiment, the lesion is a polyp in the large-intestine mucosa. The type of the lesion is a type of the polyp classified by the pit pattern of microvessels in the mucosa of the polyp.

According to the present embodiment, the detection process of detecting the position of the polyp in the large-intestine mucosa is performed at the normal magnification, and the diagnosis process of diagnosing the type of the polyp is performed at the greater magnification. As a result, it is possible, in the pit pattern diagnosis using the large-intestine endoscope, to appropriately provide the diagnostic support in the normal observation and the diagnostic support in the enlarged observation.

Further, in the present embodiment, the first trained model 121 is trained, using the first annotation 631b indicating the position of the lesion in the first training image 631a, to detect the position of the lesion indicated by the first annotation 631b from the first training image 631a. In the detection process, the processing device 110 detects the position of the lesion from the target image IMIN. The second trained model 122 is trained, using the second annotation 632b indicating the type of the lesion in the second training image 632a, to detect the type of the lesion indicated by the second annotation 632b from the second training image 632a. The processing device 110 diagnoses the type of the lesion from the target image IMIN in the diagnosis process.

According to the present embodiment, there are generated the first trained model 121 for detecting the lesion position from the image at the normal magnification and a second trained model 122 for diagnosing the lesion type from the image at the greater magnification. As a result, the processing using the first trained model 121 implements the detection process of detecting the lesion position from the target image IMIN at the normal magnification, and the processing using the second trained model 122 implements the diagnosis process of diagnosing the lesion type from the target image IMIN at the greater magnification.

Further, in the present embodiment, the processing device 110 acquires in the diagnosis process the diagnosis result together with reliability of the diagnosis result, and performs processing of displaying the diagnosis result and the reliability on the display device 400.

The reliability is an index indicating certainty of the diagnosis result, and is exemplified by a score output by a neural network, for example. According to the present embodiment, presentation of the diagnosis result and the reliability to the user as diagnostic support information allows the user to evaluate the diagnosis result on the basis of the reliability. As a result, the user can easily perform more appropriate diagnosis.

### 3. Detailed Example of Normal-Magnification Detection Process and Greater-Magnification Diagnosis Process

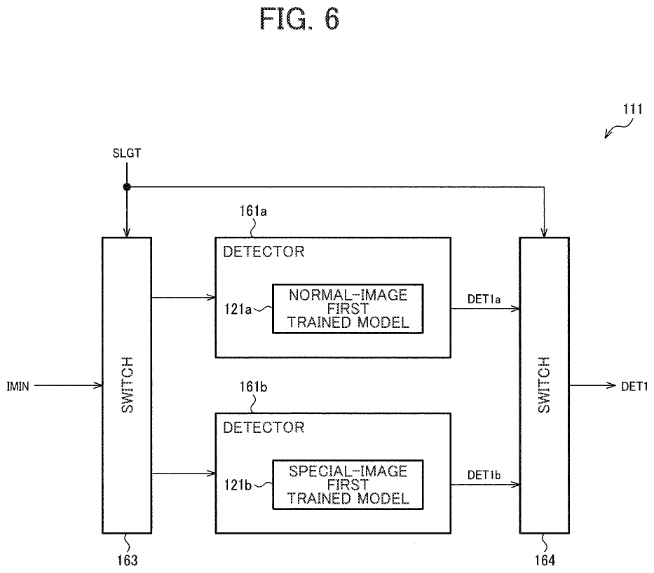

FIG. 6 shows a first detailed example of the normal-magnification detection process 111.

In this example, observation mode information SLGT indicating an observation mode set by the operation device 220 is input to the processing device 110. When image recognition is performed in real time, the observation mode information SLGT is input from the operation device 220 to the processing device 110. Alternatively, when image recognition is performed ex post facto after completion of imaging of the inside of the living body, the storage device 120 temporarily records the target image IMIN and the observation mode information SLGT, and the recorded target image IMIN and the observation mode information SLGT are input to the processing device 110. For example, the storage device 120 records a video, metadata for each frame image of the video is recorded, and the metadata contains the observation mode information SLGT.

The observation mode includes a normal observation mode and a special observation mode, which indicate kinds of illumination light or whether to involve staining. The normal observation mode is a mode in which an image of the inside of the living body is captured under the white light condition and unstained condition. The image captured in the normal observation mode will be referred to as the normal image. The special observation mode is a mode in which an image of the inside of the living body is captured under the white light condition and stained condition, under the special light condition and unstained condition, or under the special light condition and stained condition. The image captured in the special observation mode will be referred to as the special image.

A switch 163 performs processing of selecting either a detector 161a or a detector 161b according to the observation mode information SLGT. The switch 163 outputs the target image IMIN to the detector 161a when the observation mode information SLGT indicates the normal observation. In this case, the target image IMIN is a normal image. In a case where the observation mode information SLGT indicates the special observation, the target image IMIN is output to the detector 161b. In this case, the target image IMIN is a special image.

The detector 161a detects the lesion from the normal image through processing using the normal-image first trained model 121a and outputs a detection result DET1a of the lesion. The detector 161b detects the lesion from the special image through processing using the special-image first trained model 121b and outputs a detection result DET1b of the lesion.

The normal-image first trained model 121a and the special-image first trained model 121b are separate trained models contained in the first trained model 121 and are trained in advance by the learning device. The normal-image first trained model 121a receives inputs of a first training normal image captured in the normal observation mode and the annotation attached thereto as training data, and is trained to be able to detect the lesion from the first training normal image. The special-image first trained model 121b receives inputs of a first training special image captured in the special observation mode and an annotation attached thereto as training data, and is trained to be able to detect the lesion from the first training special image.

A switch 164 performs processing of selecting either the detection result DET1a or the detection result DET1b according to the observation mode information SLGT. The switch 164 outputs the detection result DET1a as the detection result DET1 to the switch 114 when the observation mode information SLGT indicates the normal observation mode, and outputs the detection result DET1b as the detection result DET1 to the switch 114 when the observation mode information SLGT indicates the special observation mode.

Although FIG. 6 shows an example of providing separate detectors for the normal image and the special image, the detection process may be performed by a single detector for the normal image and the special image. In either case where the target image IMIN is the normal image or the special image, the target image IMIN is input to the detector, and the detector detects the lesion from the target image IMIN through processing based on the first trained model 121. In this case, the first trained model 121 is not provided separately for the normal image and the special image, and is a single trained model. The first trained model 121 receives inputs of the first training normal image captured in the normal observation mode, the annotation attached thereto, the first training special image captured in the special observation mode, and the annotation attached thereto as training data, and is trained to be able to detect the lesion from the first training normal image and the first training special image.

In the present embodiment as described above, the target image IMIN is the normal image captured under the white light condition, or the special image captured under at least one of the special light condition and stained condition. The first training image 631a includes the first training normal image captured under the white light condition and the first training special image captured under at least one of the special light condition and stained condition. The first trained model 121 includes the normal-image first trained model 121a trained using the first training normal image and the special-image first trained model 121b trained using the first training special image. The processing device 110 detects, when the normal image is input, the lesion from the target image IMIN through processing based on the normal-image first trained model 121a, and detects, when the special image is input, the lesion from the target image IMIN through processing based on the special-image first trained model 121b.

According to the present embodiment, the trained model used for the detection process is switched between the normal image and the special image. In other words, it is possible to prepare a trained model corresponding to appearance in the normal image of organs, tissues, mucosal textures, or the like and a trained model corresponding to appearance in the special image of organs, tissues, mucosal textures, or the like, and to use these models for the detection process in a switching manner. This makes it possible to perform diagnostic support capable of dealing with various tasks that combine the observation using the normal image with the observation using the special image.

Further, in the present embodiment, the first trained model 121 may be trained using the first training normal image and the first training special image.

According to the present embodiment, without providing separate trained models, i.e., the normal-image first trained model 121a and the special-image first trained model 121b, a single trained model can implement the detection process on the normal image and the special image.

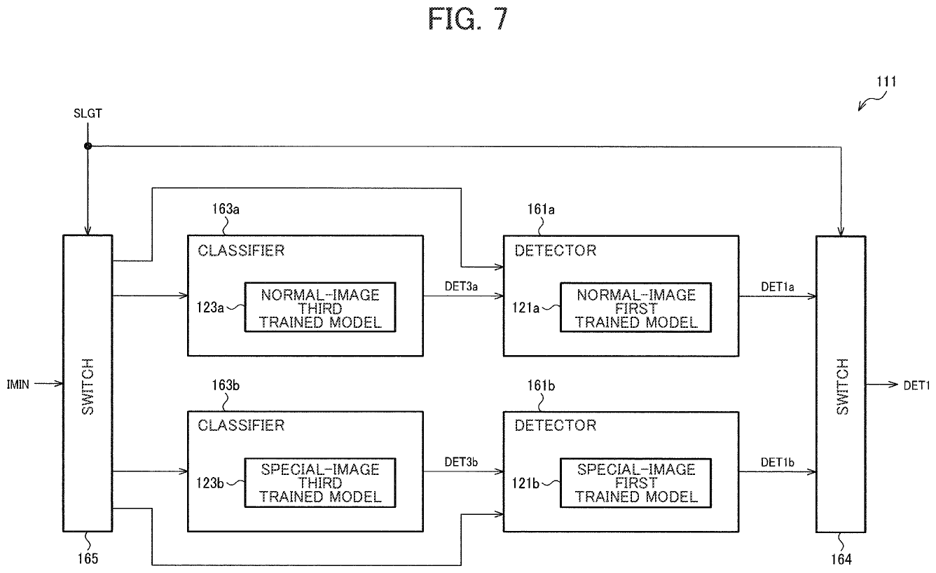

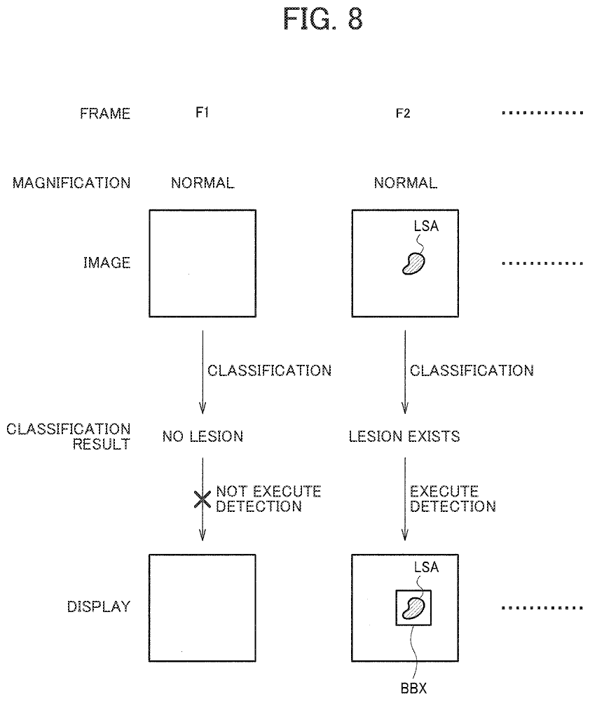

FIG. 7 shows a second detailed example of the normal-magnification detection process 111. Further, FIG. 8 shows an explanatory diagram of processing performed by the normal-magnification detection process 111 in the second detailed example. As shown in FIG. 7, in the second detailed example, classifiers 163a and 163b are provided in preceding stages of the detectors 161a and 161b, respectively, and operation of the detectors 161a and 161b is adjusted on the basis of the classification results by the classifiers 163a and 163b.

A switch 165 performs processing of selecting either the classifier 163a and the detector 161a, or the classifier 163b and the detector 161b according to the observation mode information SLGT. The switch 163 outputs the target image IMIN to the classifier 163a and the detector 161a when the observation mode information SLGT indicates the normal observation. If the observation mode information SLGT indicates the special observation, the target image IMIN is output to the classifier 163b and the detector 161b.

The classifier 163a performs a classification process on the normal image through processing using a normal-image third trained model 123a and outputs a classification result DET3a of the normal image. The classifier 163b performs a classification process on the special image through processing using a special-image third trained model 123b and outputs a classification result DET3b of the special image. The normal-image third trained model 123a and the special-image third trained model 123b are stored in the storage device 120 of the information processing system 100 as a third trained model. The classification process here is processing of determining whether the target image IMIN to be input is an image containing the lesion or an image not containing the lesion. In the classification process, a score indicating certainty is obtained, the certainty indicating that the image is an image containing the lesion, and when the obtained score is equal to or greater than a threshold value, the image is determined to be the image containing the lesion.

The normal-image third trained model 123a and the special-image third trained model 123b are separate trained models and are trained in advance by the learning device. To the normal-image third trained model 123a, the third training normal image captured in the normal observation mode and the annotation attached thereto are input as training data. The annotation indicates whether or not the third training normal image is an image containing the lesion. The normal-image third trained model 123a is trained to be able to determine whether or not the third training normal image is an image containing the lesion using the above-mentioned training data. To the special-image third trained model 123b, the third training special image captured in the special observation mode and the annotation attached thereto are input as training data. The annotation indicates whether or not the third training special image is an image containing the lesion. The special-image third trained model 123b is trained to be able to detect the lesion from the third training special image using the above-mentioned training data.

The detector 161a adjusts the detection process according to the classification result DET3a. As shown in FIG. 8, it is assumed that, in the frame F1, the classifier 163a has determined that the target image IMIN contains no lesion. When receiving an input of the classification result DET3a indicating that the target image IMIN contains no lesion, the detector 161a does not perform the processing of detecting the lesion from the target image IMIN. It is assumed that, in the frame F2, the classifier 163a has determined that the target image IMIN contains the lesion LSA. When receiving an input of the classification result DET3a indicating that the target image IMIN contains the lesion, the detector 161a performs processing of detecting the lesion from the target image IMIN. The detector 161a generates the boundary box BBX indicating the position of the lesion LSA, and the generated boundary box BBX is superimposed and displayed on the image.

Although FIG. 7 shows an example of providing two sets of classifiers and detectors for the normal image and the special image, processing may be performed with a single set of a classifier and a detector for the normal image and the special image. In either case where the target image IMIN is the normal image or the special image, the target image IMIN is input to the classifier and the detector. The classifier determines whether or not the target image IMIN is an image containing the lesion through processing based on the third trained model. In this case, the third trained model is not provided separately for the normal image and the special image, and is a single trained model. The third trained model receives inputs of the third training normal image captured in the normal observation mode, the annotation attached thereto, the third training special image captured in the special observation mode, and the annotation attached thereto as training data, and is trained to be able to detect whether or not the third training normal image and the third training special image are images containing the lesion. The detector performs the detection process on the target image IMIN through processing based on the first trained model. At this time, the detector adjusts the detection process on the target image IMIN according to the classification result.

In the present embodiment as described above, the storage device 620 stores the third trained model. The third trained model is a trained model that has been trained, using the third training image at the normal magnification, to classify whether or not the third training image is an image containing the lesion. When the magnification change information SMAG indicates the normal magnification, the processing device 110 classifies whether or not the target image IMIN is an image containing the lesion through processing based on the third trained model. When determining that the target image IMIN is an image containing the lesion, the processing device 110 performs the detection process on the target image IMIN. Note that in FIG. 7, the normal-image third trained model 123a and the special-image third trained model 123b correspond to the third trained model. Alternatively, without separately providing trained models for the normal image and the special image, a single trained model may constitute the third trained model.

According to the present embodiment, using the third trained model allows execution of classification as to whether or not the target image IMIN at the normal magnification is an image containing the lesion. As a result, the detection process on the target image IMIN can be adjusted according to the classification result. For example, when the target image IMIN is determined to contain the lesion, the detection process is executed on the target image IMIN.

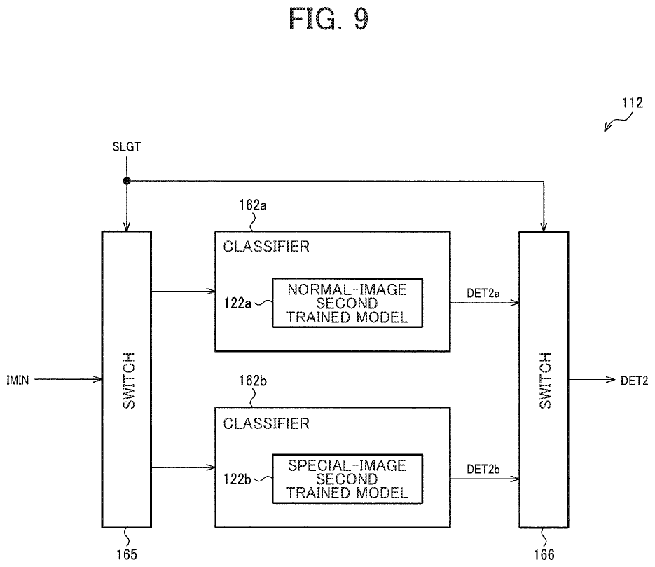

FIG. 9 shows a detailed example of a greater-magnification diagnosis process 112.

The switch 165 performs processing of selecting either a classifier 162a or a classifier 162b according to the observation mode information SLGT. The switch 165 outputs the target image IMIN to the classifier 162a when the observation mode information SLGT indicates the normal observation. When the observation mode information SLGT indicates the special observation, the target image IMIN is output to the classifier 162b.

The classifier 162a diagnoses the lesion from the normal image through processing using the normal-image second trained model 122a, and outputs a diagnosis result DET2a of the lesion. The classifier 162a diagnoses the lesion from the special image through processing using the special-image second trained model 122b and outputs a diagnosis result DET2b of the lesion.

The normal-image second trained model 122a and the special-image second trained model 122b are separate trained models contained in the second trained model 122 and are trained in advance by the learning device. The normal-image second trained model 122a receives inputs of a second training normal image captured in the normal observation mode and the annotation attached thereto as training data, and is trained to be able to diagnose the lesion from the second training normal image. The special-image second trained model 122b receives inputs of a second training special image captured in the special observation mode and the annotation attached thereto as training data, and is trained to be able to diagnose the lesion from the second training special image.

A switch 166 performs processing of selecting either the diagnosis result DET2a or the diagnosis result DET2b according to the observation mode information SLGT. The switch 166 outputs the diagnosis result DET2a as the diagnosis result DET2 to the switch 114 when the observation mode information SLGT indicates the normal observation mode, and outputs the diagnosis result DET2b as the diagnosis result DET2 when the observation mode information SLGT indicates the special observation mode.

Although FIG. 9 shows an example of providing separate classifiers for the normal image and the special image, the diagnosis process may be performed using a single classifier for the normal image and the special image. In either case where the target image IMIN is the normal image or the special image, the target image IMIN is input to the classifier, and the classifier diagnoses the lesion from the target image IMIN through processing based on the second trained model 122. In this case, the second trained model 122 is not provided separately for the normal image and the special image, and is a single trained model. The second trained model 122 receives inputs of the second training normal image captured in the normal observation mode, the annotation attached thereto, the second training special image captured in the special observation mode, and the annotation attached thereto as training data, and is trained to be able to detect the lesion from the second training normal image and the second training special image.

In the present embodiment as described above, the target image IMIN is a normal image captured under the white light condition, or a special image captured under at least one of the special light condition and stained condition. The second training image 632a includes the second training normal image captured under the white light condition and the second training special image captured under the at least one of the special light condition or stained condition. The second trained model 122 includes the normal-image second trained model 122a trained using the second training normal image and the special-image second trained model 122b trained using the second training special image. The processing device 110 diagnoses, when the normal image is input, the type of the lesion from the target image IMIN through processing based on the normal-image second trained model 122a, and diagnoses, when the special image is input, the type of the lesion from the target image IMIN through processing based on the special-image second trained model 122b.

According to the present embodiment, the trained model used for the diagnosis process is switched between the normal image and the special image. In other words, it is possible to prepare a trained model corresponding to appearance in the normal image of organs, tissues, mucosal textures, or the like and a trained model corresponding to appearance in the special image of organs, tissues, mucosal textures, or the like, and to use these models for the diagnosis process in a switching manner. This makes it possible to perform diagnostic support capable of dealing with various tasks that combine the observation using the normal image with the observation using the special image.

Further, in the present embodiment, the second trained model may be trained using the second training normal image and the second training special image.

According to the present embodiment, without providing separate trained models, i.e., the normal-image second trained model 122a and the special-image second trained model 122b, a single trained model can implement the diagnosis process on the normal image and the special image.

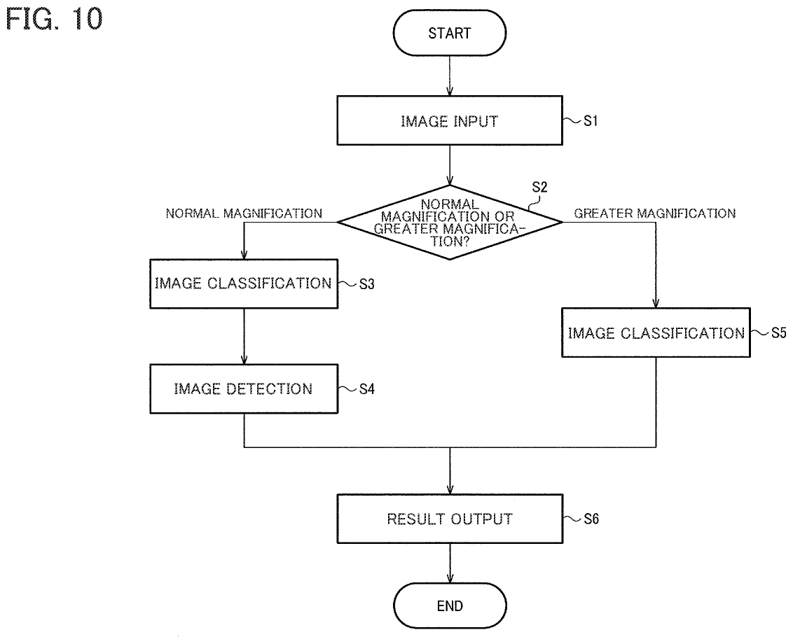

FIG. 10 shows a flowchart of processing performed by the processing device 110.

In step S1, the target image IMIN is input to the processing device 110. In step S2, the processing device 110 determines whether the magnification of the target image IMIN is the normal magnification or the greater magnification on the basis of the magnification change information SMAG.

When the magnification is determined to be the normal magnification in step S2, the processor 110 determines, in step S3, whether or not the target image IMIN is an image containing the lesion by the classifier 163a or the classifier 163b. In step S4, the processing device 110 detects the lesion from the target image IMIN by the detector 161a or the detector 161b.

When the magnification is determined to be the greater magnification in step S2, the processor 110 diagnoses, in step S5, the lesion from the target image IMIN by the classifier 162a or the classifier 162b.

In step S6, the processing device 110 generates the display image IMDS by superimposing the detection result of step S4 or the diagnosis result of step S5 on the target image IMIN, and outputs the generated display image IMDS to the display device 400.

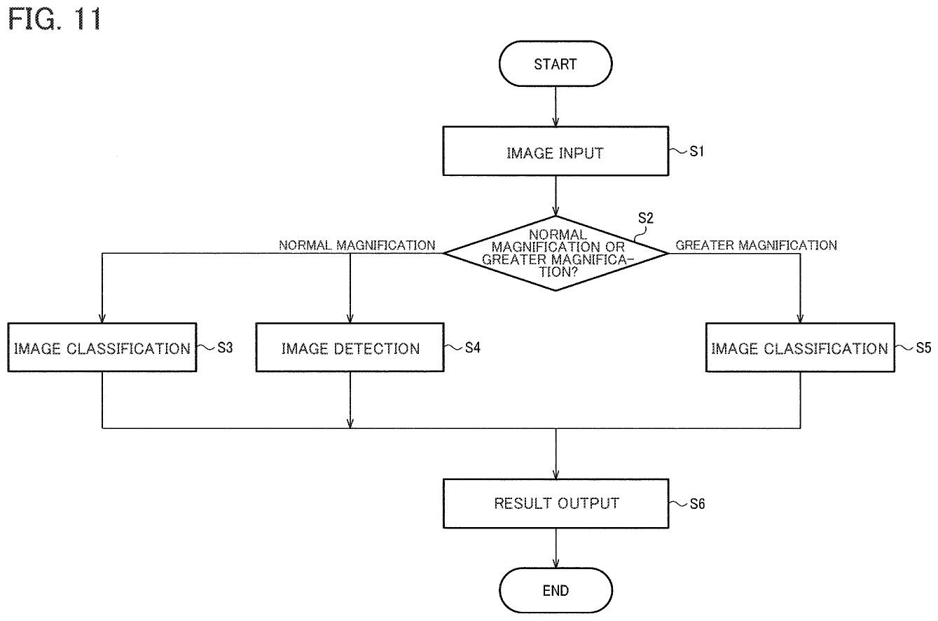

Although the above description provides an example in which classification and detection are executed in series in the normal-magnification detection process 111, classification and detection may be executed in parallel in the normal-magnification detection process 111 as shown in FIG. 11. When the magnification is determined to be the normal magnification in step S2 of FIG. 11, the processing device 110 performs the classification process in step S3 and the detection process in step S4 in parallel. That is, the detection process in step S4 is executed independent of the result of the classification process in step S3. In step S6, the processing device 110 adjusts and displays the result of the detection process in step S4 according to the result of the classification process in step S3. That is, when determining that the target image IMIN contains the lesion in step S3, the processing device 110 displays the boundary box generated by the detection process in step S4. When determining in step S3 that the target image IMIN contains no lesion, the processing device 110 does not display the boundary box generated by the detection process in step S4.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. An information processing system comprising:
a memory storing a first trained model and a second trained model; and
a processor configured to perform a detection process and a diagnosis process, the detection process being a process of detecting a lesion from a target image through processing based on the first trained model, the target image being captured by an imaging device of an endoscope system and input to the processor, the diagnosis process being a process of diagnosing a type of the lesion from the target image through processing based on the second trained model,
wherein the first trained model is a trained model that has been trained, using a first training image having a normal magnification, to perform the detection process on the first training image,
the second trained model is a trained model that has been trained, using a second training image that has a greater magnification than the normal magnification, to perform the diagnosis process on the second training image, and
magnification change information being input to the processor from an operation device of the endoscope system configured to change magnification of the target image, the processor is configured to perform the detection process on the target image when the magnification change information indicates the normal magnification, and perform the diagnosis process on the target image when the magnification change information indicates the greater magnification.

2. The information processing system as defined in claim 1, wherein
a normal image being the target image at the normal magnification is an image captured under a first condition, the first condition comprising at least one of white light condition, special light condition, and stained condition,
the first trained model is trained using the first training image captured under the first condition,
a special image being the target image at the greater magnification is an image captured under a second condition, the second condition comprising at least one of special light condition and stained condition, the special image being an enlarged part of a field of view of the normal image, and
the second trained model is trained using the second training image captured under the second condition.

3. The information processing system as defined in claim 1, wherein
each of the target image at the normal magnification and the target image at the greater magnification is an image captured under special light condition and unstained condition, and
the greater magnification is 50 times or more and less than 100 times the normal magnification in terms of observation magnification on a display screen.

4. The information processing system as defined in claim 3, wherein
a spectrum of the special light has a peak belonging to a bandwidth of 390 nm to 445 nm and a peak belonging to a bandwidth of 530 nm to 550 nm.

5. The information processing system as defined in claim 3, wherein
the lesion is a polyp in a large-intestinal mucosa, and
the type of the lesion is a type of the polyp classified by a pit pattern of microvessels in the mucosa of the polyp.

6. The information processing system as defined in claim 1, wherein
the first trained model is trained, using a first annotation indicating a position of the lesion in the first training image, to detect the position of the lesion indicated by the first annotation from the first training image,
the processor detects the position of the lesion from the target image in the detection process,
the second trained model is trained, using a second annotation indicating a type of the lesion in the second training image, to detect the type of the lesion indicated by the second annotation from the second training image, and
the processor diagnoses the type of the lesion from the target image in the diagnosis process.

7. The information processing system as defined in claim 6, wherein
the processor performs processing of acquiring a diagnosis result together with reliability of the diagnosis result in the diagnosis process and displaying the diagnosis result and the reliability on a display device.

8. The information processing system as defined in claim 1, wherein
the target image is a normal image captured under white light condition or a special image captured under at least one of special light condition and stained condition,
the first training image includes a first training normal image captured under the white light condition and a first training special image captured under the at least one of the special light condition and the stained condition,
the first trained model includes a normal-image first trained model that has been trained using the first training normal image and a special-image first trained model that has been trained using the first training special image, and
the processor detects, when the normal image is input, the lesion from the target image through processing based on the normal-image first trained model, and detect, when the special image is input, the lesion from the target image through processing based on the special-image first trained model.

9. The information processing system as defined in claim 1, wherein
the target image is a normal image captured under white light condition or a special image captured under at least one of special light condition and stained condition,
the first training image includes a first training normal image captured under the white light condition and a first training special image captured under the at least one of the special light condition and the stained condition, and
the first trained model is trained using the first training normal image and the first training special image.

10. The information processing system as defined in claim 1, wherein
the target image is a normal image captured under white light condition or a special image captured under at least one of special light condition and stained condition,
the second training image includes a second training normal image captured under the white light condition and a second training special image captured under the at least one of the special light condition and the stained condition,
the second trained model includes a normal-image second trained model that has been trained using the second training normal image and a special-image second trained model that has been trained using the second training special image, and
the processor diagnoses, when the normal image is input, the type of the lesion from the target image through processing based on the normal-image second trained model, and diagnose, when the special image is input, the type of the lesion from the target image through processing based on the special-image second trained model.

11. The information processing system as defined in claim 1, wherein
the target image is a normal image captured under white light condition or a special image captured under at least one of special light condition and stained condition,
the second training image includes a second training normal image captured under the white light condition and a second training special image captured under at least one of the special light condition and the stained condition, and
the second trained model is trained using the second training normal image and the second training special image.

12. The information processing system as defined in claim 1, wherein
the memory stores a third trained model,
the third trained model is a trained model that has been trained, using a third training image having the normal magnification, to classify whether or not the third training image is an image containing the lesion, and
the processor classifies, when the magnification change information indicates the normal magnification, whether or not the target image is the image containing the lesion through processing based on the third trained model, and perform, when determining that the target image is the image containing the lesion, the detection process on the target image.

13. An endoscope system comprising:
the information processing system as defined in claim 1;
the imaging device; and
the operation device.

14. An information processing method comprising:
inputting a target image captured by an imaging device of an endoscope system and magnification change information acquired by an operation device of the endoscope system that changes magnification of the target image,
performing a detection process when the magnification change information indicates a normal magnification, the detection process being a process of detecting a lesion from the target image through processing based on a first trained model that has been trained, using a first training image having the normal magnification, to detect the lesion from the first training image, and
performing a diagnosis process when the magnification change information indicates a greater magnification than the normal magnification, the diagnosis process being a process of diagnosing a type of the lesion from the target image through processing based on a second trained model that has been trained, using a second training image having the greater magnification, to diagnose a type of the lesion from the second training image.
